(19) United States
(12) Patent Application Publication
(10) Pub. No.: US 2002/0188354 A1
(43) Pub. Date: Dec. 12, 2002

(54) Device to treat obesity by obstructing gastric outlet

(76) Inventor: Paolo Lino Peghini, Rehoboth Beach, DE (US)

Correspondence Address: Paolo L. Peghini, 67 Kings Creek Circle, Rehoboth Beach, DE, 19971, US

(21) Appl. No.: 09/878,958

(22) Filed: Jun. 12, 2001

(51) Int. Cl. A61F 2/04

(52) U.S. Cl. 623/23.65; 606/157; 623/23.67

(57) ABSTRACT

Obstructing the gastric outlet by inserting a foreign body into it will result in loss of appetite and weight loss.

### BACKGROUND

Obesity resulting from inadequate oral calorie intake is a condition associated with several deleterious effects to health. A device resulting in reduced oral calorie intake will cause weight loss. Physiologically ingested food pools in the stomach, from where it is gradually delivered through the pylorus (small opening between the stomach and duodenum) into the intestinal tract. Filling of the stomach is an important mechanism of satiety. Patients with poor gastric emptying from mechanical or neural conditions loose their appetite, feel uncomfortable after eating and loose weight.

This observation is the base of the here presented invention. A foreign body is inserted to obstruct the passage of stomach contents into the small bowel.

### DESCRIPTION OF THE PRIOR ART

Not applicable

### SUMMARY

Obstruction of the gastric outlet (pylorus: junction between the stomach and the small bowel) by insertion of a foreign body though the pyloric opening reduces appetite and causes weight loss. A sandglass shaped device, being placed in such a manner that it extends through the pylorus, will result in delayed gastric emptying and cause the person to loose weight. The device is made of some sort of plastic and filled with a medium, adding or removal of which allows adjustment of the size/diameter of the device and therefore the degree of obstruction of gastric emptying and its effect on appetite and food intake.

### DRAWINGS

Not Applicable

### DETAILED DESCRIPTION

The balloon-like device is made of some sort of plastic. In its expanded state it has a sandglass like shape. It has two bulged parts with a narrow connecting bridge between them. This shape is achieved by using plastics of different compliance. In its collapsed state it is placed endoscopically on a delivery catheter in such a manner that the waist (narrow part) lies in the pyloric opening.

It is then filled so that the bulges and the connecting bridge expand. The medium used to fill it is either a liquid or gas. The device can have radioopaque markers (stripes or dots) which allow radiographic control of the position or alternatively the filling medium could be radioopaque.

One of the bulges lies distal to the pylorus in the small bowel. The other one lies proximal in the stomach. These bulges keep the device in place.

This device obstructs the pylorus partially, thereby inhibiting gastric emptying causing retention of food and a sensation of fullness, resulting in decreased appetite and consequently decreased caloric intake and loss of weight.

Filling of the device with medium occurs at the time when it is placed endoscopically through the placing catheter. A connector is then loosened allowing the placing catheter to be withdrawn with the device being left in place.

Alternatively, the device could have a small catheter, which is placed during the endoscopy in such a manner that it exits from the nose, thereby providing access to the device, allowing adjustment of the size of the device by adding or removing filling medium after placement of the device.

Also, the device could be constructed in such a manner, that it contains a reservoir of medium and a remotely controlled pump, which would allow adjusting its size by pumping medium from the reservoir into the device.

What I claim as my invention is:

1. That obstruction of the gastric outlet by inserting a foreign body through its opening (pylorus) will cause weight loss.

2. That alteration of the size of the pylorus-obstructing device allows adjustment of the rapidity and extent of weight loss.
